(19) United States
(12) Patent Application Publication
(10) Pub. No.: US 2021/0065132 A1
(43) Pub. Date: Mar. 4, 2021

(54) EMPLOYER MARKETS

(71) Applicant: Synchrony Bank, Stamford, CT (US)

(72) Inventors: Sally Yijing Yang, Stamford, CT (US); Craig Urbansky, Stamford, CT (US); Kendra Larsen, Stamford, CT (US); Clarinda Gurr, Stamford, CT (US); Vishal Sriram, Stamford, CT (US); Erin Gadhavi, Stamford, CT (US)

(73) Assignee: Synchrony Bank, Stamford, CT (US)

(21) Appl. No.: 17/003,096

(22) Filed: Aug. 26, 2020

Related U.S. Application Data
(60) Provisional application No. 62/891,544, filed on Aug. 26, 2019.

(51) Int. Cl. G06Q 10/10; G06Q 40/02; G16H 40/20; G06Q 20/20; G06Q 20/40; G06Q 30/04; G06Q 40/08

(52) U.S. Cl. CPC G06Q 10/1057; G06Q 40/025; G16H 40/20; G06Q 40/08; G06Q 20/4037; G06Q 30/04; G06Q 20/202

(57) ABSTRACT

The present disclosure relates generally to payment methods. In one example, the systems and methods described herein may integrate three separate infrastructures to provide payment for medical services: the employer's benefits and compensation platform, the health savings account provider's platform, and the financing platform.

The present application claims benefit of U.S. provisional application Ser. No. 62/891,544, filed Aug. 26, 2019 the disclosure of which is incorporated herein by reference.

### FIELD

### Field

The present disclosure relates generally to payment methods. In one example, the systems and methods described herein may integrate three separate infrastructures to provide payment for medical services: the employer's benefits and compensation platform, the health savings account provider's platform, and the financing platform.

### SUMMARY

Currently, many employers offer health benefits to their active employees. This includes various medical insurance plan options, including High Deductible Health Plans (HDHPs). These HDHPs allow the consumer (i.e., subscriber) to pay a lower monthly fee compared to other medical plan options, but have a high insurance deductible for each benefit year. This is a great option if the consumer does not expect to use his health insurance; however, unexpected health events can incur large, and immediate, out-of-pocket expenses as a result of the high deductible. Depending on the consumer's financial capability and cash liquidity, this can result in the consumer being unable to meet his financial responsibility post-procedure, or prevent them from seeking treatment in the first place.

The systems and methods described herein address these needs and others by the combination of: (1) reducing monthly insurance premiums by allowing a consumer to enroll in a high deductible health plan (HDHP), (2) utilizing HealthCredit credit functionality to pay out-of-pocket costs (i.e., a deductible) owed to his healthcare provider upfront, (3) utilizing HealthCredit financing options to distribute repayment responsibility (to HealthCredit) across a defined period of months, (4) utilizing automatic health savings account (HSA) or paycheck deductions to avoid late payments, and (5) utilizing insurance to cover healthcare expenses above the maximum deductible amount.

To achieve this, the systems and methods described herein may define the integration of three separate infrastructures: (1) the employer's benefits and compensation platform, (2) the HSA provider's platform, and (3) the financing platform. This integration may allow for the real-time flow of data across systems including, but not limited to, employee benefits selections, employee enrollment and terminations, insurance data, financial transactions, credit availability, HSA and paycheck withdrawals, and settlement activity.

The systems and methods described herein may further enable the consumer to activate these services through a single benefits enrollment. The consumer may only need to enroll through his employer at the start of the benefit year or at a life event initiating the simultaneous enrollment of medical insurance and credit/financing.

The systems and methods described herein may further utilize individual and aggregated health data to predict optimum future states. Through machine learning, the systems described herein may support a recommendation engine, providing new enrollees with personalized information to help them best take advantage of their employer's available health benefits. Through data and machine learning, the system may also restrict/protect enrollees from overextending themselves beyond their financial capacity to repay debt in a timely manner. The user may have the option to fund monthly repayment of promotional financing through his HSA account or paycheck withholding, ensuring timely repayment of debt. Further, the user may also have the ability to fund monthly repayment of promotional financing through a bank transfer from its bank account. Upon completion, the consumer may receive his account(s) necessary to validate his eligibility for each service via a single swipe/key of card information at point of transaction.

According to some embodiments, a computer-program product is provided. The computer-program product is tangibly embodied in a non-transitory machine-readable storage medium, including instructions that, when executed by one or more processors, cause the one or more processors to perform the steps of the above method.

According to some embodiments, a system is provided. The system comprises one or more processors, and one or more non-transitory machine-readable storage media containing instructions that, when executed on the one or more processors, cause the one or more processors to perform operations including the steps of the above method.

This summary is not intended to identify key or essential features of the claimed subject matter, nor is it intended to be used in isolation to determine the scope of the claimed subject matter. The subject matter should be understood by reference to appropriate portions of the entire specification of this patent application, any or all drawings, and each claim.

The foregoing, together with other features and examples, will be described in more detail below in the following specification, claims, and accompanying drawings.

### BRIEF DESCRIPTION OF THE DRAWINGS

Illustrative embodiments are described in detail below with reference to the following figures.

FIG. 1 depicts a flow diagram for a first state of processing for medical expenses, according to some embodiments.

FIG. 2 depicts a flow diagram of a new state of processing for medical expenses, according to some embodiments.

FIG. 3 depicts a flow diagram of how a backend system processes medical enrollment alongside financial/credit applications. It also may enable automatic debt repayment according to the systems and methods described herein, according to some embodiments.

FIG. 4 depicts a flow diagram of how machine learning is implemented in the systems and methods described herein, according to some embodiments.

FIG. 5 depicts an exemplary table of credit availability for an individual, according to some embodiments.

FIG. 6 depicts an exemplary interface 600 provided by the HealthCredit service to cardholders, according to some embodiments.

FIG. 7 depicts a computing system architecture including various components in electrical communication with each other using a connection in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

In the following description, for the purposes of explanation, specific details are set forth in order to provide a thorough understanding of certain inventive embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

Disclosed embodiments may provide a framework in which employees enrolled in high deductible health plans (HDHPs) may finance healthcare costs, in some embodiments, to the limit of the high deductible. Thus, for a plan with a $3,000 deductible, a procedure costing $6,000 can be paid by $3,000 in insurance and $3,000 in financing. The financing may be repaid automatically from the employee's paycheck or HSA account over a period of time, in some embodiments. In some embodiments, an employee may only be approved to finance a certain amount based on his or her income, debts, or any other factors. In some embodiments, the systems and methods described herein may further integrate health savings accounts (HSAs) and/or flexible spending accounts (FSAs) in order to further process medical expense payments. Although described herein as relating to medical expenses and payment sources relating to medical expenses, it is contemplated that the systems and methods described herein may be used in any context to leverage a combination of disparate payment sources and make intelligent decisions about which payment source to use and to what extent.

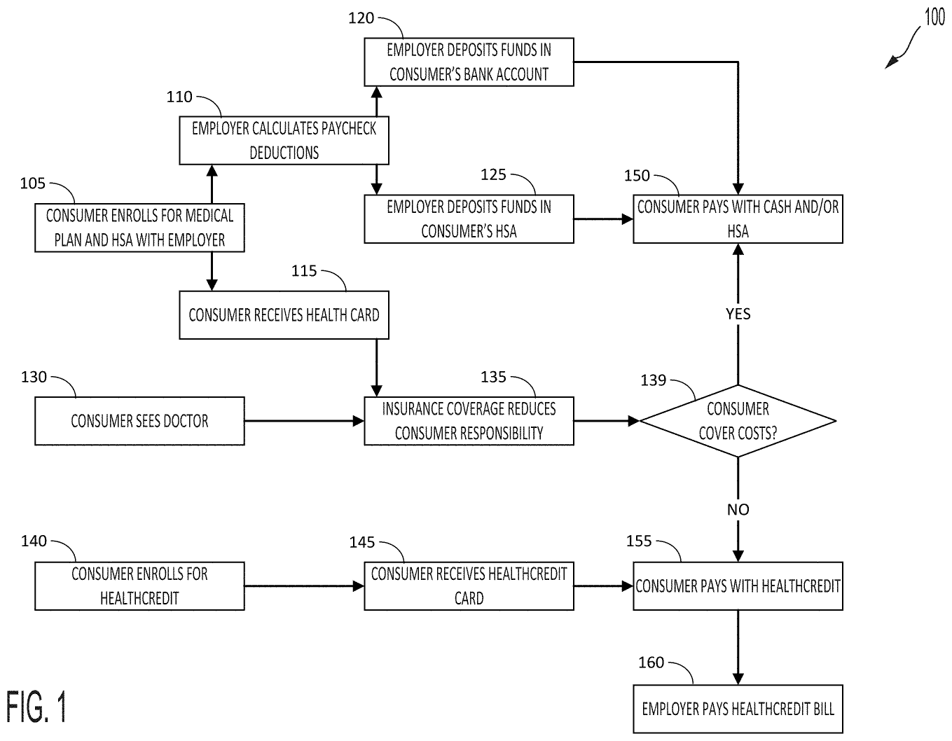

FIG. 1 depicts a flow diagram 100 for a first state of processing for medical expenses, according to some embodiments. At step 140, a consumer enrolls for HealthCredit. As used herein, “HealthCredit” may be used to refer to any credit card program designed or able to be used to pay for and/or finance medical expenses and that is backed by an employer, which may repay any balances using deductions from a cardholder's paycheck or from other cardholder's holdings (e.g., HSAs, etc.). In some instances, to enroll for HealthCredit, the consumer may be required to complete an application for a line of credit for use in payment of medical expenses. Using information supplied in the application, a detailed (e.g., hard) credit worthiness check may be performed to determine whether the consumer can be approved for a HealthCredit line of credit and, if so, the amount of the line of credit. In some instances, the application for enrollment for HealthCredit may be provided by the consumer's employer. For instance, during an enrollment period for enrollment in a medical plan provided by the employer or during a new employee onboarding process, the consumer may be presented with an opportunity to apply for a HealthCredit line of credit. Alternatively, the consumer may independently apply for enrollment for HealthCredit, such as through a website associated with HealthCredit or at any location that makes available applications for enrollment for HealthCredit (e.g., medical office, hospital, pharmacy, etc.). In the application, the consumer may be required to provide its employer information. This employer information may be used to identify any employer-defined parameters, as described herein, for determining what line of credit may be offered to the consumer (e.g., based on maximum deductible for the consumer's medical plan, based on the consumer's credit history and/or score, etc.).

In an embodiment, an employer provides, to an employer management sub-system implemented by a HealthCredit service, medical plan information and participating employee information that can be used to determine the lines of credit that may be offered to participating employees. The medical plan information may specify the various deductibles corresponding to in-network versus out-of-network procedures, medical consultations, prescriptions, and the like. Further, the medical plan information may be divided based on the different medical plans offered to different classes of participating employees. For instance, a particular class of employee (e.g., union-represented employees, employees in a particular business unit, employees in a particular location or facility, non-management employees, etc.) may be offered one or more medical plans that may not be offered to other classes of employees. The participating employee information may specify information regarding each employee participating in a medical plan and/or HSA. For instance, for a particular employee, the participating employee information may specify which medical plan the employee is enrolled in, the amount of any HSA contributions made to the employee's HSA per paycheck, the employee's salary, identifying information of the employee (e.g., name, address, etc.), and the like. This participating employee information may be used to define a maximum percentage of the employee's paycheck that can be allocated to healthcare needs versus other needs, such as food, rent, and the like.

In an embodiment, the employer management sub-system implemented by the HealthCredit service can provide the employer with an option to offer, to employees of the employer, lines of credit determined based on the deductibles defined in the medical plans offered to these employees. For instance, if a particular medical plan has a maximum deductible of $10,000, the employer management sub-system may provide the employer with an option to offer employees enrolled in the particular medical plan an opportunity to apply for a $10,000 line of credit. In an embodiment, the employer management sub-system can additionally, or alternatively, provide the employer with an option to generate individual offers for each employee based on information associated with the employee. For instance, using information associated with an employee (e.g., birthdate, last four digits of a Social Security number, etc.), the employer management sub-system may perform a soft inquiry of the employee's credit report to determine whether the employee can be pre-approved for a line of credit that can be used for medical expenses. The amount of the line of credit may be determined based on the medical plan selected by the employee, the results of the soft inquiry of the employee's credit report, the employee's salary information, and the like. For instance, if the maximum deductible of the employee's medical plan is greater than the maximum line of credit amount that can be offered to the employee, the employer management sub-system may provide the employer with an option to allow the employee to apply for a line of credit corresponding to the maximum line of credit amount. Alternatively, if the maximum deductible of the employee's medical plan is less than the maximum line of credit amount that can be offered to the employee, the employer management sub-system may provide the employer with an option to allow the employee to apply for a line of credit corresponding to the maximum deductible amount. In some instances, the employer may forego this determination and define a default maximum line of credit amount that may be offered to its employees.

If an employee (e.g., consumer) opts to apply for a HealthCredit line of credit, the application for the HealthCredit line of credit may be provided to a HealthCredit payment instrument issuer, which may evaluate the application to determine whether the consumer is approved for a HealthCredit line of credit. If the consumer is approved for a HealthCredit line of credit, the HealthCredit payment instrument issuer may determine the amount of this line of credit. For instance, if the employer has determined that the amount of the line of credit is to be equal to the maximum deductible for the employee's medical plan, the HealthCredit payment instrument issuer may determine whether the employee qualifies for a line of credit in this amount. As another example, if the employer has determined that employees may be offered lines of credit that do not exceed their maximum deductibles, the HealthCredit payment instrument issuer may determine the amount of the line of credit that the employee is qualified for and indicate, to the employee, that it is approved for a line of credit in the determined amount. At step 145, if the consumer is approved for a HealthCredit line of credit, the consumer is issued a HealthCredit credit card associated with the employer and receives this HealthCredit credit card.

In an embodiment, the HealthCredit payment instrument issuer utilizes a machine learning model or artificial intelligence trained using unsupervised learning techniques to provide the employee with a forecast of possible medical expenditures for the upcoming enrollment period. For instance, in the application for the HealthCredit line of credit, an employee may be prompted to indicate whether it is anticipating any events that may have an impact on the employee's medical expenditures over the upcoming enrollment period (e.g., birth of a child, an operation, etc.). The HealthCredit payment instrument issuer may use this information from the employee's application, as well as any available information corresponding to the employee's historical medical expenses and credit worthiness over prior enrollment periods, as input to a machine learning model or artificial intelligence to predict the employee medical expenses for the upcoming enrollment period and provide recommendations for establishing a HealthCredit line of credit, establishing and funding an HSA, and the like.

In an embodiment, if the HealthCredit payment instrument issuer determines that the employee is new to the employer (e.g., the employee was recently hired and is enrolling in a medical plan for a first time with the employer, etc.), the HealthCredit payment instrument issuer can utilize the employee's responses in the application for a HealthCredit line of credit as input to the machine learning model or artificial intelligence to provide the employee with a recommendations for establishing a HealthCredit line of credit, establishing and funding an HSA, and the like. The machine learning model or artificial intelligence may use the provided information to identify a corresponding cluster of other employees that may be similarly situated (e.g., have enrolled in a similar medical plan, have experienced medical events similar to those anticipated by the employee, are within a similar geographic area with similar cost of living and expenses, etc.). From this cluster, the machine learning model or artificial intelligence may forecast the employee's likely future medical expenses and, based on these future medical expenses, provide the employee with recommendations for managing these future medical expenses. This may include recommending establishment of a line of credit with the HealthCredit service, recommending an amount to contribute to an HSA per pay cycle, recommending how much to save from the employee's paycheck in addition to contributions to an HSA and maintaining a HealthCredit line of credit, and the like.

The machine learning model or artificial intelligence utilized by the HealthCredit payment instrument issuer may trained using unsupervised learning techniques, as noted above. For instance, a dataset of input employee information for various employees (including, but not limited to, medical expenses per employee over one or more enrollment periods, demographic information per employee, salary information per employee, allocation of funds into HSA and implementation of HealthCredit lines of credit per employee, etc.) may be analyzed using a clustering algorithm to identify estimated medical expenditures per enrollment period for certain types of employees and the types of accounts established for these employees (e.g., HSAs and corresponding funding amounts, HealthCredit lines of credit amounts, etc.). Example clustering algorithms that may be trained using this dataset provided by the employer to forecast an employee's future medical expenses and recommend HealthCredit lines of credit and/or HSAs may include k-means clustering algorithms, fuzzy c-means (FCM) algorithms, expectation-maximization (EM) algorithms, hierarchical clustering algorithms, density-based spatial clustering of applications with noise (DBSCAN) algorithms, and the like. Based on the output of the machine learning model or artificial intelligence, the HealthCredit payment instrument issuer may determine whether to extend a line of credit to the employee, the amount of the line of credit, and whether the employee should supplement their line of credit with an HSA to cover any medical expenses that may exceed the line of credit.

Meanwhile, at step 105, the consumer enrolls for a medical plan and an HSA account with his employer. For instance, the consumer's employer may provide the consumer with an opportunity to enroll in a medical plan and an HSA account when the consumer begins its employment with the employer. Further, the consumer's employer may provide the consumer with an opportunity at the start of a new enrollment period (e.g., each year, etc.) to change their enrollment in a medical plan, enroll in a new medical plan, establish an HSA account, change contributions to an existing HSA account, and the like. At step 115, the consumer receives a health insurance card associated with the selected medical plan and the consumer's HSA account (if established). At step 110, based on the consumer's selections for a medical plan and HSA contributions, the employer calculates the paycheck deductions for the HSA and the medical benefits associated with the selected medical plan. At step 120, the employer deposits funds from the consumer's paycheck, minus any paycheck deductions described above for its medical plan and HSA contributions, in the consumer's bank account. Further, at step 125, the employer deposits the funds deducted from the consumer's paycheck and indicated by the consumer as contributions to its HSA account in the consumer's HSA account.

At step 130, the consumer sees a doctor or otherwise engages in an action that requires presentation of its health insurance card (e.g., visits a hospital for a medical procedure, obtains a prescription from a pharmacy, etc.) or use of one or more medical insurance plan benefits. At step 135, the consumer presents its health insurance card, which is used to determine the consumer's insurance coverage and to reduce the patient responsibility based on the consumer's insurance coverage. For instance, a first transaction using the provided health insurance card can be used identify policy data and insurance coverage in real-time (reducing the consumer's out-of-pocket costs) and allow the consumer to determine the remaining balance. As an illustrative example, when the consumer presents its health insurance card, an insurance system associated with the medical plan may access a patient database, an insurance policy database, and pricing tables to determine the consumer's insurance coverage and, based on the aforementioned information, the consumer's patient responsibility. The consumer's patient data can be identified by the system automatically and/or in real-time with presentation of the health insurance card (e.g., swipe, key entry, scan, etc.) at the point-of-sale (e.g., doctor's office, hospital, pharmacy, etc.). The insurance policy database is used to determine whether the consumer has insurance coverage and the extent of the insurance coverage or determine if further review of the medical claim is required. Pricing tables can be used to determine patient (e.g., consumer) responsibility (e.g., to adjudicate claims in real-time). Based on this evaluation, the insurance system may determine the remaining costs that are not covered by its insurance coverage and present the consumer with these remaining costs. For instance, at a point-of-sale, the insurance system may display, to the consumer, the remaining balance that is to be paid for the medical expense.

At decision block 139, it is determined whether the consumer can cover the remaining costs not covered by the insurance coverage (e.g., the remaining balance) with its HSA account and/or with cash (e.g., fiat currency, from the consumer's bank account, etc.). For instance, the HealthCredit service may access the consumer's HSA account to determine whether the funds maintained in the HSA account are sufficient to cover the remaining costs. Alternatively, the consumer may opt to access its HSA account to pay the remaining costs not covered by the insurance coverage. Alternatively, the consumer may determine that it will pay these remaining costs using cash or a line of credit (e.g., credit card). If it is determined that the HSA account may be used to cover the remaining costs, the consumer may be provided with an option to utilize its HSA account to cover the remaining costs. In some instances, the insurance system may provide the consumer with an option to pay these remaining costs using cash (e.g., fiat currency, a credit card other than a HealthCredit credit card, etc.). If the consumer can cover the remaining costs using its HSA account and/or with cash, the consumer may opt to pay the outstanding medical bill with cash and/or with an amount from its HSA at step 150 at the point-of-sale (e.g., doctor's office, hospital, pharmacy, etc.).

If the consumer cannot cover the remaining costs with its HSA account or wishes to use its employer-backed HealthCredit account to cover the remaining costs, the consumer may opt to pay the outstanding medical bill with its HealthCredit account at step 155. For instance, the consumer may provide its employer-backed HealthCredit credit card at the point-of-sale (e.g., doctor's office, hospital, pharmacy, etc.) to apply the remaining costs to the HealthCredit line of credit. In some instances, the consumer may opt to pay the remaining costs using any combination of its HSA account, cash, and its HealthCredit credit card account. As an illustrative example, the consumer may indicate that the HSA account may be used to pay for a portion of the remaining costs and that the remainder is to be paid using other payment methods. The consumer, thus, may opt to pay this portion of the remaining costs with its HSA account and the remaining portion with a combination of cash and its HealthCredit credit card account. This may allow the consumer to tailor payment of any remaining medical expenses to its financial needs.

At step 160, the employer repays any debt incurred on the consumer's HealthCredit account. For instance, a HealthCredit billing system may provide the employer with a bill specifying any medical expenses incurred by the consumer over a most recent billing cycle and paid using the consumer's HealthCredit line of credit. Further, the bill may specify the consumer's current balance, which may include the medical expenses incurred by the consumer during the most recent billing cycle and any previous balances incurred by the consumer. The employer, based on the consumer's current balance, may deduct a particular amount (subject to a maximum percentage of the consumer's paycheck that can be allocated to healthcare needs versus other needs) from the consumer's paycheck for the payment of at least a minimum portion of the current balance. In some instances, any remaining balance may be deducted from the consumer's paycheck over a period of time subject to the maximum percentage of the consumer's paycheck that can be allocated to healthcare needs.

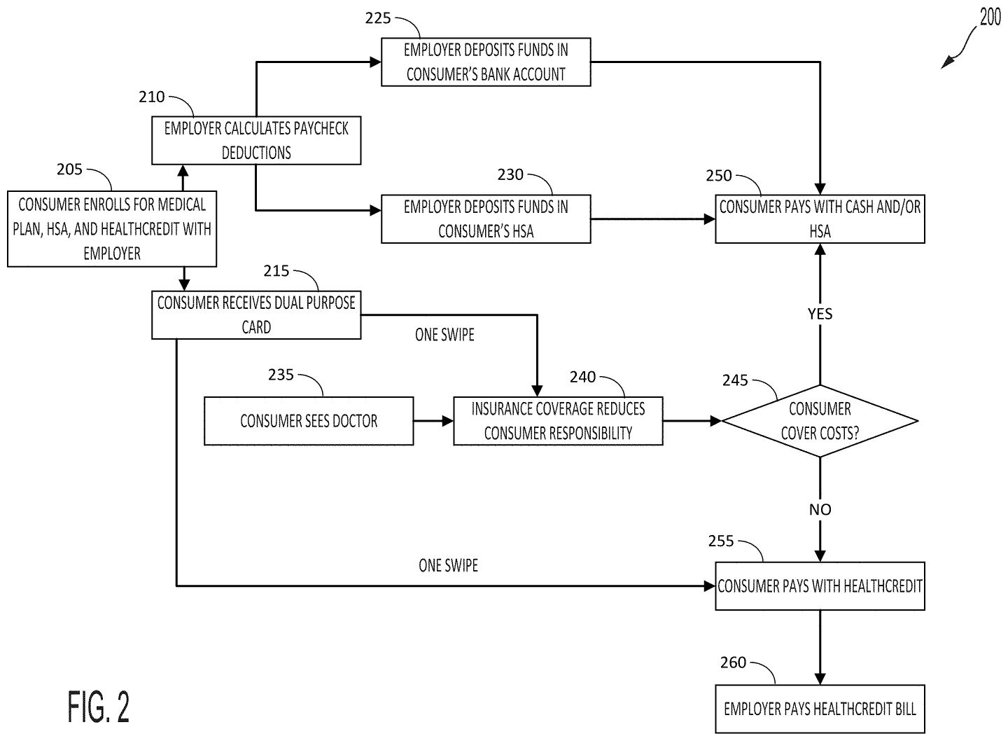

FIG. 2 depicts a flow diagram 200 of a second state of processing for medical expenses, according to some embodiments. At step 205, the consumer enrolls for a medical plan and a HealthCredit line of credit with its employer. For instance, the consumer may be provided with an option to apply for more than one service (e.g., both insurance and credit) at the same time via its employer. When the consumer applies for a HealthCredit line of credit via its employer, an employer management sub-system of the HealthCredit service may determine any spending caps for the consumer. For instance, the employer management sub-system may determine the consumer's spending cap based on the consumer's income, debts, and historical information of similarly situated individuals and historical information about the consumer. In some instances, the employer management sub-system of the HealthCredit service may perform a detailed (e.g., hard) credit worthiness check to determine whether the consumer can be approved for a HealthCredit line of credit and, if so, the amount of the line of credit. The employer management sub-system may also utilize information provided by the employer to determine a spending cap or line of credit for the consumer. For instance, the employer management sub-system may utilize the consumer's payment schedule and amount, as provided by the employer, to determine the consumer's spending cap or line of credit.

As noted above, the employer management sub-system implemented by the HealthCredit service can provide the employer with an option to offer, to employees of the employer, lines of credit determined based on the deductibles defined in the medical plans offered to these employees. For instance, an employer may determine that the amount of a line of credit for an employee is to be equal to that of the maximum deductible of the employee's selected medical plan. Thus, the employer management sub-system may determine, based on the information provided by the consumer, whether the consumer qualifies for a line of credit in the amount equal to that of the maximum deductible of the consumer's medical plan. If the consumer does not qualify for this line of credit, the employer management sub-system may recommend that the consumer should establish an HSA or supplement an existing HSA with additional funds for its medical expenses.

In some instances, the employer may allow an employee (e.g., consumer) to apply for a HealthCredit line of credit that has an amount less than the maximum deductible of the employee's medical plan. For instance, using information associated with an employee (e.g., birthdate, last four digits of a Social Security number, etc.), the employer management sub-system may perform a soft inquiry of the employee's credit report to determine whether the employee can be pre-approved for a line of credit that can be used for medical expenses. The amount of the line of credit may be determined based on the medical plan selected by the employee, the results of the soft inquiry of the employee's credit report, the employee's salary information, and the like. For instance, if the maximum deductible of the employee's medical plan is greater than the maximum line of credit amount that can be offered to the employee, the employer management sub-system may provide the employer with an option to allow the employee to apply for a line of credit corresponding to the maximum line of credit amount. Alternatively, if the maximum deductible of the employee's medical plan is less than the maximum line of credit amount that can be offered to the employee, the employer management sub-system may provide the employer with an option to allow the employee to apply for a line of credit corresponding to the maximum deductible amount.

In an embodiment, the employer management sub-system uses available underwriting data to identify a maximum percentage of the consumer's paycheck that can be allocated to healthcare needs versus other needs, such as food, rent, and the like. This information may be provided to the consumer, which may optionally choose to have its employer pay his future financing debt via the consumer's HSA or from the consumer's paycheck. In some instances, the information regarding the possible allocation of a consumer's paycheck to healthcare needs may additionally, or alternatively, be provided to the employer. If the consumer opts to obtain a HealthCredit line of credit, the employer (subject to the consumer's approval) may determine a maximum amount that may be deducted from the consumer's paycheck and/or HSA to pay off any line of credit debts. This maximum amount may be subject to the maximum percentage of the consumer's paycheck that can be allocated to healthcare expenses and prior to any disbursement of funds to the consumer's HSA account or bank account.

At step 215, the consumer receives a health insurance card and an employer-backed HealthCredit credit card. In some embodiments, the health insurance card and employer-backed HealthCredit credit card may be implemented as a single card. In such embodiments, the single card may be swiped or keyed once at a point-of-sale to access the consumer's medical plan and the consumer's HealthCredit line of credit. The single card may be used in a single transaction at the point-of-sale to identify policy data and insurance coverage in real-time (reducing the consumer's out-of-pocket costs) and to allow the consumer to submit payment for the remaining balance using any combination of its HSA account, HealthCredit line of credit, and cash (e.g., fiat currency, other lines of credit, etc.), as described above. Having access to both policy data (e.g., insurance policy data) and credit data (e.g., cardholder credit data) allows the the HealthCredit service to apply funds (e.g., for insurance covered items, an insurance reimbursement), to an outstanding balance at the point-of sale or directly to the consumer's account shortly thereafter, automatically, and without cardholder intervention or interaction.

At step 210, the employer calculates paycheck deductions for HSA, medical benefits, and any spending on the employer-backed HealthCredit account. This calculation may be based at least in part on the information supplied by the employer management sub-system defining the maximum percentage of the consumer's paycheck that can be allocated to healthcare needs versus other needs, such as food, rent, and the like, as described above. At step 220, the employer withholds funds for HealthCredit spending (e.g., repayment of any debts associated with the consumer's HealthCredit line of credit) and for the consumer's HSA account from the consumer's paycheck. At step 225, the employer deposits funds from the consumer's paycheck into the consumer's bank account minus any amounts reserved for allocation to the consumer's HSA account, and any other elected allocations (e.g., medical plan expenses, union fees, etc.). At step 230, the employer deposits funds allocated by the consumer for its HSA account into the consumer's HSA account.

With a single swipe or key of the card issued to the consumer and that is associated with the consumer's medical plan and HealthCredit line of credit, a consumer can utilize more than one service (e.g., medical plan insurance, HealthCredit account, any other line of credit, etc.), in a single transaction (e.g., a single swipe of a card, a single data entry, a single scan, etc.) at a point-of-sale. By using the multi-purpose card issued to the consumer, the consumer can also avoid having to separately submit a bill for reimbursement. For instance, when the card is utilized at the point-of-sale, an insurance system associated with the consumer's medical plan may identify, in real-time, the consumer's medical plan and determine the consumer's payment responsibility. The consumer may be provided with its remaining balance at the point-of-sale and the consumer may determine whether to pay this remaining balance using its HSA account, its employer-backed HealthCredit line of credit, or cash/other lines of credit. Thus, a single transaction can concurrently pay for a medical service and determine whether to apply credit or financing at the time of payment for the service. A singular transaction can eliminate the inconvenience of having to perform transactions separately. Additionally, having access to both policy data (e.g., insurance policy data) and credit data (e.g., cardholder credit data), allows the employer management sub-system to apply credits or refunded amounts (e.g., from an insurance re-imbursement), to an outstanding balance at the point-of sale or shortly thereafter, automatically, and without cardholder intervention or interaction.

Meanwhile, at step 235, the consumer sees a doctor. At step 240, the consumer swipes its dual-purpose health insurance/employer-backed HealthCredit card at the point-of-sale (e.g., doctor's office, hospital, pharmacy, etc.) to submit the consumer's information to the insurance system associated with the consumer's medical plan. The insurance system may use the consumer's information to identify the consumer's medical plan and determine the insurance coverage for the visit with the doctor. Based on the consumer's medical plan, the insurance system reduces the consumer's payment responsibility. In some instances, the insurance system may present the consumer with its payment responsibility at the point-of-sale.

At decision block 245, it is determined whether the consumer can cover the costs unpaid by insurance using cash or its HSA. For instance, the consumer may determine whether it wishes to use its HSA account pay for the unpaid costs. Additionally, or alternatively, the consumer may determine, at the point-of-sale, whether to pay the unpaid costs using cash (e.g., fiat currency, a debit card, an alternative credit card, etc.). If the consumer can cover the costs (e.g., the consumer has sufficient funds in its HSA account for the unpaid costs, etc.), the consumer may use its HSA or cash to pay the outstanding medical bill at step 250.

In some instances, the consumer may be presented with an option to pay the outstanding medical bill using different payment methods. For example, if the HSA does not have sufficient funds to pay for all of the unpaid costs, the consumer may opt to pay some of the unpaid costs using the funds available in the HSA and the remainder using cash and/or its employer-backed HealthCredit line of credit. Thus, the consumer may tailor its payment of the unpaid costs using any combination of its HSA, cash, and its HealthCredit line of credit.

If the consumer determines that it cannot cover the unpaid costs using its HSA or the consumer wishes to use its HealthCredit line of credit to cover the unpaid costs, the consumer may opt to pay the outstanding medical bill with its HealthCredit line of credit at step 255. For instance, the consumer may be provided, at the point-of-sale, with an option to utilize its HealthCredit line of credit to cover part or all of the unpaid costs. This may allow the consumer to determine how to allocate the unpaid costs among its HSA, its HealthCredit line of credit, and/or other cash payment method. Further, if the consumer does not have sufficient funds in its HSA and HealthCredit line of credit, the consumer may determine what additional funds are required to cover any remaining unpaid costs. Thus, at the point-of-sale, the consumer may customize how any unpaid cost is to be paid using any of its HSA, HealthCredit line of credit, and other payment method.

At step 260, if the consumer opts to pay the unpaid medical costs using its HealthCredit line of credit, the employer pays the HealthCredit bill with funds withheld from the employee's HSA or paycheck for use in paying debts incurred on the consumer's HealthCredit line of credit. For instance, the employer management sub-system may provide, to the employer, the consumer's HealthCredit bill detailing any balances incurred as a result of payment of medical expenses using the consumer's HealthCredit line of credit. In some instances, if additional funds are required to pay the HealthCredit bill, the employer may utilize funds from the consumer's HSA account to pay the HealthCredit bill. Additionally, or alternatively, the employer may pay the HealthCredit bill and deduct any amount paid not using the consumer's HSA and funds withheld for payment of HealthCredit balances from one or more future paychecks subject to a maximum percentage of the consumer's paycheck that can be allocated to healthcare needs.

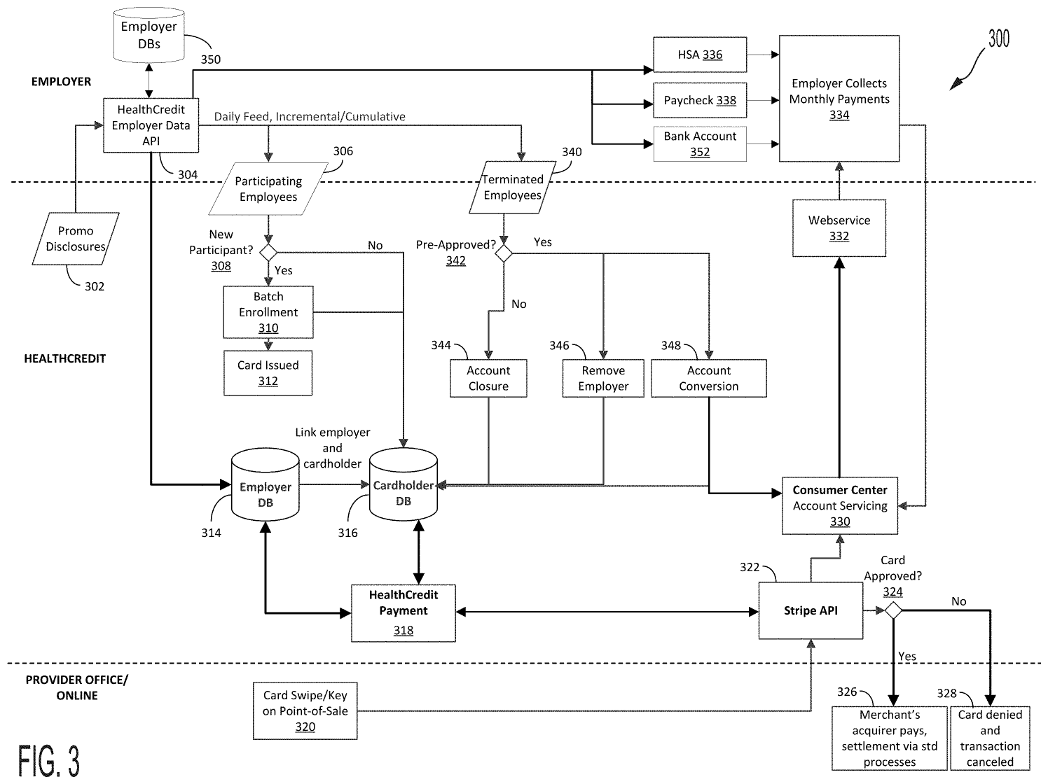

FIG. 3 depicts a flow diagram 300 of how a backend system of the HealthCredit service processes medical expenses according to the systems and methods described herein, according to some embodiments. The backend system of the HealthCredit service may comprise one or more components, as described herein, that may collectively process information from an employer (e.g., employee information, medical insurance plan benefit information, HSA contribution information, etc.) to generate and maintain lines of credit for the employees of the employer. At step 302, promotional disclosures are made by the HealthCredit service. For instance, when an employer submits a request to the HealthCredit service to enroll in a credit program whereby employees of the employer may establish lines of credit with the HealthCredit service for payment of medical expenses, the HealthCredit service may provide the employer with one or more promotional disclosures. The promotional disclosures may specify a set of promotional interest rates for lines of credit that may be offered to an employer's employees. Additionally, or alternatively, the promotional disclosures may specify any benefits that may be provided, on a promotional basis, to the employer's employees, such as bonus loyalty rewards points, cash back benefits, gift cards, and the like.

At step 304, the employer management sub-system of the HealthCredit service is directed to an application programming interface (API) for employer data. The employer management sub-system may be implemented using a computer system or as an application or other executable code implemented on a computer system of the HealthCredit service. The API may be provided with a daily feed that is either incremental or cumulative from one or more employer databases 350 maintained by the employer. These employer databases 350 may include employee information, medical insurance plan information, annual or enrollment period spending cap information, and the like. The daily feed may specify participating employees 306 that have enrolled in HealthCredit to establish a line of credit provided by HealthCredit for use in paying medical expenses. At step 308, the employer management sub-system of the HealthCredit service determines whether a participating employee is a new participant. For instance, the employer management sub-system may evaluate employment data for each employee specified in the daily feed. This employment data for a particular employee may specify the employment date (e.g., first day of employment) of the employee. Further, the employment data may specify the date in which the employee enrolled in a medical plan or made changes to its medical plan, such as during an enrollment period for making medical plan elections. In some instances, the employee data may specify whether the employee has submitted an application for a line of credit with the HealthCredit service. For instance, the employee data may include the submitted application for the employee. Alternatively, the employee data may specify a network address from which the employee's application for a line of credit with the HealthCredit service may be retrieved. Thus, from the employee data, if the employer management sub-system identifies a new participant that wishes to establish a line of credit with the HealthCredit service, the employer management sub-system may obtain the employee's application for this line of credit, as well as other information that may be used to determine whether the employee can be approved for this line of credit.

If the employee is a new participant, the employer management sub-system performs batch enrollment of the newly participating employees may be made at step 310. For instance, the employer management sub-system may evaluate the application submitted by each newly participating employee to determine whether a line of credit may be extended to the newly participating employee and, if so, the amount of this line of credit. For instance, the employer management sub-system may utilize the information provided in the completed application to perform a detailed (e.g., hard) credit worthiness check to determine whether the newly participating employee can be approved for the preferred payment instrument and, if so, the amount of the line of credit. In an embodiment, the employer management sub-system garners employee information from the employee data supplied by the employer. For instance, the employee data may specify the newly participating employee's income or salary, the geographic location or address of the employee, any HSA contributions per paycheck established by the employee, the parameters of the medical insurance plan in which the employee is enrolled (e.g., maximum deductibles, paycheck deductions for enrollment in the medical insurance plan, etc.), any paycheck deductions for other employment-related expenses (e.g., union dues, fitness center memberships, employer meal plan contributions, 401K or other retirement plan contributions, etc.), and the like. Using this employee data, the employer management sub-system may calculate the maximum percentage of the employee's paycheck that may be used for medical expenses, including repayment of any debts incurred against a line of credit with the HealthCredit service.

As noted above, an employer may define what lines of credit may be offered to its employees. For instance, an employer may define a requirement whereby an employee can be offered a line of credit in an amount equal to the maximum deductible defined in the employee's current medical insurance plan. In some instances, the employer may define a requirement whereby an employee can be offered a line of credit in an amount that is up to the maximum deductible defined in the employee's current medical insurance plan (e.g., the line of credit may be in an amount less than or equal to the maximum deductible amount). The requirement may be defined on a per-employee basis, on a per-employee class (e.g., union represented, non-management, location-specific, etc.) basis, and the like. Thus, the employer management sub-system may determine whether the employer has defined such requirements for a newly participating employee and, if so, utilize this requirement as part of its determination of whether the newly participating employee can be approved for a line of credit with the HealthCredit service.

At step 312, a card may be issued to newly participating employees approved for a line of credit with the HealthCredit service. The card may be backed by a third-party payment processor or other financial institution that may process transactions relying on the line of credit provided by the HealthCredit service. The card may be utilized as a financial instrument (e.g., credit card, debit card, etc.) at a point-of-sale exclusively for medical expenses. For instance, the issued card may be utilized at a point-of-sale associated with a medical service (e.g., hospital, doctor's office, pharmacy, etc.) and for expenses keyed, or otherwise identified, as being tied to a medical service. Thus, any use of the issued card for expenses other than those identified as being for medical services may be automatically denied. In some instances, the card may be used to access multiple services, such as the employee's HealthCredit line of credit, the employee's HSA, and the like. In some instances, rather than issuing a physical card, the HealthCredit service may enable the employee to access its HealthCredit line of credit using a digital wallet that may be associated with its HSA and medical insurance plan.

In an embodiment, if the employer management sub-system determines that an employee is not approved for a line of credit with the HealthCredit service, the employer management sub-system transmits a notification to the employer to indicate that the particular employee has been denied a line of credit with the HealthCredit service. This may cause the employer to inform the employee of the denial of the line of credit. In some instances, if an employee is denied a line of credit with the HealthCredit service, the employer management sub-system may provide one or more recommendations to the employee for managing its medical expenses. For instance, the employer management sub-system may use a machine learning model or artificial intelligence trained using unsupervised learning techniques to provide the employee with a forecast of possible medical expenditures for the upcoming enrollment period. For instance, in the application for the HealthCredit line of credit, an employee may be prompted to indicate whether it is anticipating any events that may have an impact on the employee's medical expenditures over the upcoming enrollment period (e.g., birth of a child, an operation, etc.). The employer management sub-system may use this information from the employee's application, as well as any available information corresponding to the employee's historical medical expenses over prior enrollment periods, as input to a machine learning model or artificial intelligence to predict the employee's medical expenses for the upcoming enrollment period and provide recommendations for establishing and funding an HSA, for saving funds for possible medical expenses, and the like. In some instances, if the employee is not approved for a line of credit with the HealthCredit service, the employer may provide its own line of credit to the employee or, alternatively, forego offering a line of credit to the employee.

If the employee is not a new participant at step 308, the employer management sub-system bypasses the aforementioned new enrollment processes for the employee. The employee's information may be retrieved or stored at a cardholder database 316. The cardholder database 316 may include closed accounts, active employee accounts, and active unemployed accounts, as well as corresponding transactions tied to these accounts. The cardholder database 316 may be in operative communication with an employer database 314. The employer database 314 may include a list of employers, annual caps as defined by these employers, benefit years (e.g., enrollment periods, etc.), and promotional settings to be applied to each employee as defined by the employer. The information stored in the employer database 314 may be provided by an employer when engaging the HealthCredit service to allow for enrollment of employees in the HealthCredit service as part of an enrollment period for medical benefits. For instance, as part of an employer enrollment process, the employer may be prompted to provide information regarding the medical insurance plans or other benefits that are provided to its employees, any spending caps that the employer may impose on its employees for medical expenses or any other contributions (e.g., retirement plan contributions, HSA contributions, etc.), its enrollment periods for different employee classes, and the like. In some instances, based on the information supplied by the employer, the employer management sub-system may identify one or more promotional benefits that may be presented to the employees of the employer as a benefit for obtaining a HealthCredit line of credit. The employer may determine which promotional benefits are to be offered to its employees, which may result in definition of a set of promotional settings as described above.

The API 304 may also provide access to terminated employees 340. For instance, the daily feed may specify which employees have been terminated or have otherwise left the employ of the employer. As an illustrative example, for a newly terminated employee, the feed may specify a termination date or the last date of employment with the employer. As noted above, an employee's line of credit with the HealthCredit service may be backed by the employer, whereby the employer may deduct certain amounts from an employee's paycheck to pay any outstanding balances on the employee's line of credit. Thus, at step 342, the employer management sub-system determines whether the terminated employee 340 is pre-approved for its current line of credit with the HealthCredit service or for a new line of credit that can replace the pre-existing line of credit established via the terminated employee's former employer. For instance, the employer management sub-system may transmit a request to an account management sub-system of the HealthCredit service to access the terminated employee's account and obtain information about the terminated employee that may be used to perform a soft credit worthiness check of the terminated employee. The account management sub-system may be implemented using a computer system or as an application or other executable code implemented on a computer system of the HealthCredit service.

If the terminated employee 340 is pre-approved for a line of credit with the HealthCredit service, the account management sub-system may access the terminated employee's account and remove the associated employer from the account at step 346. Further, at step 348, the account management sub-system may convert the existing account for the terminated employee into a personal account, subject to new terms and conditions, statements, and billing. For instance, the account management sub-system may execute a new detailed credit worthiness check of the terminated employee to determine whether the terminated employee is approved for the line of credit previously established with the HealthCredit service through its former employer. In an embodiment, the account management sub-system provides the terminated employee with a new application for the line of credit previously established with the HealthCredit service (e.g., through the terminated employee's e-mail address, physical address, etc.). In the new application, the terminated employee may specify its new level of income and other information (e.g., new employer, new salary, etc.) that may be used to determine whether the terminated employee can be approved for the previously established line of credit with the HealthCredit service. If, based on the information supplied by the terminated employee or otherwise garnered via evaluation of the terminated employee's account, the account management sub-system determines that the terminated employee is approved for the previously established line of credit, the account management sub-system may provide the terminated employee with any new terms and conditions applicable to the account. For instance, the new terms and conditions may indicated that the terminated employee is solely responsible for repayment of any outstanding balances. The terms and conditions may also specify alternative interest rates, as the promotional interest rates previously applicable to the account may be specific to the employer and are no longer applicable.

In an embodiment, if the terminated employee is not approved for the previously established line of credit, the account management sub-system transmits a notification to the terminated employee to provide payment for any outstanding balance on the account. For instance, the account management sub-system may provide the terminated employee with a bill detailing the outstanding balance and its requirement to repay the outstanding balance in a lump sum payment or over a pre-determined period of time as established by the HealthCredit service. In some instances, if the account associated with the terminated employee is tied to a bank account of the terminated employee, the account management sub-system may automatically withdraw the remaining balance from this bank account if repayment is not provided within a predetermined period of time.

In some instances, if the terminated employee is approved for a line of credit in an amount that is less than the amount for the previously established line of credit, the account management sub-system may transition an outstanding balance to the new line of credit and request that the terminated employee repay any amount of the outstanding balance that could not be applied to the new line of credit. In some instances, if the terminated employee wishes to close the account and forego maintaining the existing line of credit or establishing a new line of credit with the HealthCredit service, the account management sub-system may provide the terminated employee with a final bill detailing any outstanding balances and prompting the terminated employee to submit payment for the outstanding balances in order for the account to be closed. If the terminated employee 340 is not pre-approved for a line of credit or, upon conducting a detailed credit worthiness check, the terminated employee 340 is not approved for the line of credit, the account may be closed at step 344. The relevant data associated with the closed account may be saved in the cardholder database 316.

At step 318, payment of a pending transaction submitted via an API 322 may occur based on information specified in the employer database 314 and the cardholder database 316, as described herein. Further, at step 318, account status and spend caps may be checked for the employee, and a promotion may be applied based artificial intelligence and machine learning. As noted above, the HealthCredit service may utilize a machine learning model or artificial intelligence trained using unsupervised learning techniques to provide an employee with a forecast of possible medical expenditures for the upcoming enrollment period. For instance, in the application for the HealthCredit line of credit, an employee may be prompted to indicate whether it is anticipating any events that may have an impact on the employee's medical expenditures over the upcoming enrollment period (e.g., birth of a child, an operation, etc.). The HealthCredit service may use this information from the employee's application, as well as any available information corresponding to the employee's historical medical expenses over prior enrollment periods, as input to a machine learning model or artificial intelligence to predict the employee's medical expenses for the upcoming enrollment period and provide recommendations for establishing a HealthCredit line of credit, establishing and funding an HSA, and the like. Similarly, the machine learning model or artificial may determine an employee's spend caps based on the employee's ability to pay back the credit based on the employee's income, debts, and historical information of similarly situated individuals and historical information about the employee.

In some instances, an employee's spend caps may be determined based on a detailed (e.g., hard) credit worthiness check of the employee. In some instances, based on the determined spend caps for the employee and the aforementioned information used to determine these spend caps, only certain plans may be recommended or enrolled in by a particular employee. This may also be based on historical factors, such as whether the employee has previously paid back lines of credit, and/or factors related to similarly situated employees. Thus, the systems and methods described herein may be used to provide financing to consumers, but particularly for low income cardholders, prevent them from incurring debt that they cannot repay and damaging their credit score. For example, annual spending may be capped through knowledge gleaned from the employer, such as the employee's pay schedule and amount.

In some instances, the employer management sub-system utilized by HealthCredit may obtain, from the employer, an employee's actual pay amount over a period of time to determine the employee's average pay amount over the period of time. This may allow the employer management sub-system to account for various factors that can impact an employee's pay amount. For example, an employee's pay amount may differ over time based on the amount of paid overtime that the employee has worked. As another example, an employee's pay amount may change as a result of a promotion or job title change. An employee's pay amount may also change as a result of a cost of living adjustment applied to the employee's salary resulting from a move to a more expensive location.

In an embodiment, the employer management sub-system utilizes the one or more machine learning algorithms or artificial intelligence to predict fluctuations in an employee's pay amount over time in order to better determine a spending cap for an employee. For instance, the employer management sub-system may utilize an employee's pay amount over a pre-determined period of time, as well as information provided by the employer with regard to its operations (e.g., hours worked by similarly situated employees over the pre-determined period of time, production metrics over the pre-determined period of time, distribution of bonuses over the pre-determined period of time, etc.), as input to one or more machine learning algorithms or artificial intelligence to obtain a spending cap for the particular employee. The one or more machine learning algorithms or artificial intelligence may be trained using supervised learning techniques. For instance, a dataset of employee-employer inputs and corresponding employee spending caps can be selected for training of the machine learning algorithms or artificial intelligence. In some examples, the inputs can be obtained from administrators of the employer management sub-system, different employers for which employee spending caps were manually determined and evaluated over time, or other sources associated with the employer management sub-system. The machine learning algorithms or artificial intelligence may be evaluated to determine, based on the sample inputs supplied to the machine learning algorithms or artificial intelligence, whether the machine learning algorithms or artificial intelligence are providing accurate spending caps for each employee. Based on this evaluation, the machine learning algorithms or artificial intelligence may be modified (e.g., one or more parameters or variables may be updated) to improve the accuracy of the machine learning algorithms or artificial intelligence in determining an appropriate spending cap for an employee based on the provided inputs for the employee and corresponding employer.

Based on underwriting data available through HealthCredit, the employer management sub-system may identify a maximum percentage of a person's paycheck that can be allocated to healthcare needs versus other basic needs, such as food, rent, and the like. The underwriting data may include demographic information for other similarly situated individuals. For instance, the underwriting data may include known salary information, medical expenses, housing expenses, household expenses (e.g., food, utilities, etc.), and the like for individuals in a particular location. This information may also be sorted according to other demographic features, such as age, race, gender, and the like. Thus, based on one or more demographic characteristics of the consumer and underwriting data corresponding to these characteristics, the maximum percentage of the person's paycheck can be identified. Guaranteed repayment of the debt may be achieved as the employer can withhold funds from the employee's paycheck to pay off the HealthCredit debt before it is accessible to the consumer via HSA or their paycheck. Further, the amount withheld from the consumer's paycheck may be determined based on the maximum percentage identified based on the underwriting data available through HealthCredit and any allocations specified by the consumer for its medical insurance plan and its HSA.

At a medical provider (e.g., hospital, doctor's office, pharmacy, etc.) or online, a card may be swiped or keyed on a point-of-sale machine for payment of medical services or other medical expenses at step 320. At step 322, a transaction monitoring sub-system of the HealthCredit service may utilize an online payment service API (e.g., Stripe API, etc.) to log the transaction activity associated with the transaction at the point-of-sale. The transaction monitoring sub-system may be implemented using a computer system or as an application or other executable code implemented on a computer system of the HealthCredit service.

At step 324, the transaction monitoring sub-system determines whether the employee's card is approved for the transaction. If the transaction monitoring sub-system determines that the transaction is approved (e.g., the remaining balance of the employee's line of credit is sufficient for the expense, etc.), a merchant acquirer associated with the provider may pay the bill immediately, and settlement may occur via standard bank card processes. However, if the transaction monitoring sub-system determines that the transaction is not approved (e.g., the remaining balance of the employee's line of credit is insufficient, the expense is not for a medical purpose, etc.), the transaction may be denied.

The transaction may be recorded in a consumer center of the HealthCredit service for account servicing at step 330. The consumer center of the HealthCredit service may be implemented as a web service at step 332. For instance, the web service 332 may allow the employer to collect monthly payment at step 334 from the HSA 336, the employee's paycheck 338, or from an employee's bank account 352 (subject to employee approval). Alternatively, if the employee does not maintain an HSA account, the web service 332 may allow the employer to directly collect monthly payment for any outstanding balances of the employee's HealthCredit line of credit from the employee's paycheck according to a maximum percentage of the employee's paycheck that can be allocated to healthcare needs, such as repayment of a HealthCredit line of credit balance, versus other basic needs, such as food, rent, and the like.

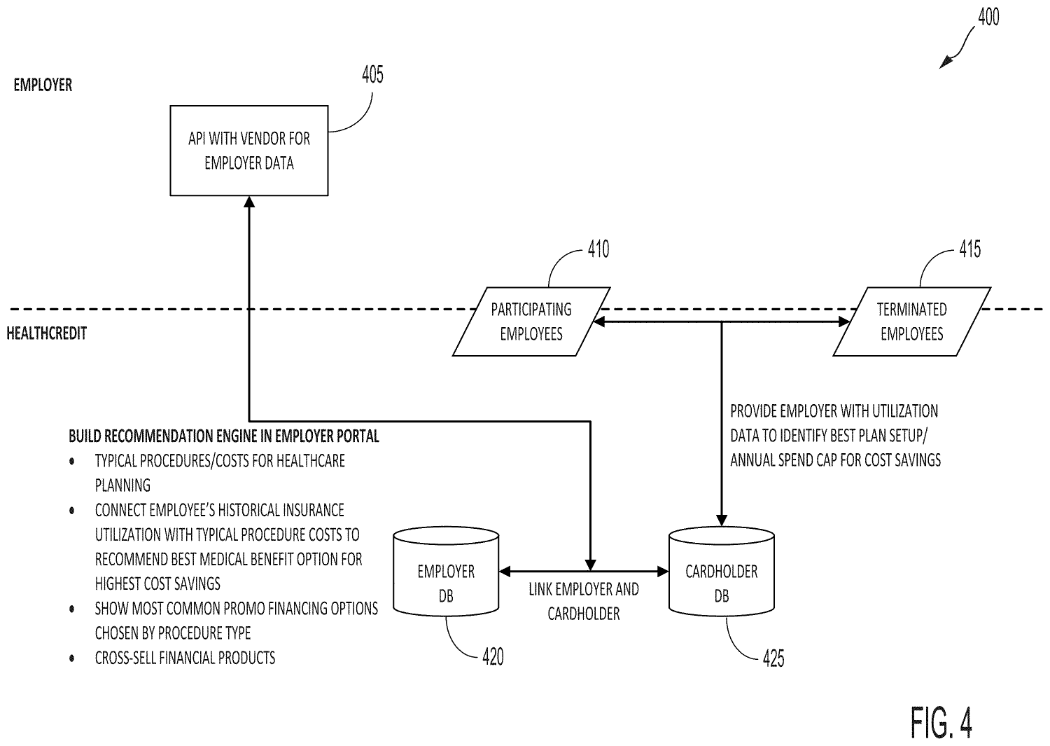

FIG. 4 depicts a flow diagram 400 of how a machine learning model or artificial intelligence is implemented in the systems and methods described herein, according to some embodiments. Information associated with participating employees 410 and terminated employees 415 may be stored in a cardholder database 425 of closed accounts, active employee accounts, and active unemployed accounts. In an embodiment, an employer management sub-system of the HealthCredit service utilizes the cardholder database 425 to provide the employer with utilization data that can be used to identify an optimal medical plan setup and annual spend cap for its employees. The identification of an optimal medical plan setup may be based on one or more factors as defined by the employer. For instance, the employer may specify that it wishes to obtain the greatest cost savings in paying for medical expenses. Further, the employer may specify that it wishes to obtain the greatest amount spending flexibility for its employees, whereby it may be ideal to provide employees with multiple funding sources (e.g., HSA, HealthCredit accounts, etc.) for payment of medical procedures.

The cardholder database 425 may be linked to the employer database 420. The employer database 420 may store an employer's annual cap, enrollment periods, and promotional settings. The information stored in the employer database 420 may be provided by an employer when engaging the HealthCredit service to allow for enrollment of employees in the HealthCredit service as part of an enrollment period for medical benefits. For instance, as part of an employer enrollment process, the employer may be prompted to provide information regarding the medical insurance plans or other benefits that are provided to its employees, any spending caps that the employer may impose on its employees for medical expenses or any other contributions (e.g., retirement plan contributions, HSA contributions, etc.), its enrollment periods for different employee classes, and the like. In some instances, based on the information supplied by the employer, the employer management sub-system of the HealthCredit service may identify one or more promotional benefits that may be presented to the employees of the employer as a benefit for obtaining a HealthCredit line of credit. The employer may determine which promotional benefits are to be offered to its employees.

The HealthCredit service may also implement a recommendation engine in the employer portal. The recommendation engine may be implemented using a computer system or as an application or other executable code implemented on a computer system of the HealthCredit service. The recommendation engine may identify and present typical procedures and costs for healthcare planning, connect an employee's historical insurance utilization with typical procedure costs to recommend the best medical benefit option for highest cost savings, show the most common promotional financing options chosen by procedure type, cross-sell financial products, and the like. In some instances, the recommendation engine may be implemented as a backend of an API with the vendor for employer data 405.

In an embodiment, the recommendation engine utilizes a machine learning model or artificial intelligence to provide the aforementioned information to employers and employees or these employers. For instance, in the application for a HealthCredit line of credit, an employee may be prompted to indicate whether it is anticipating any events that may have an impact on the employee's medical expenditures over the upcoming enrollment period (e.g., birth of a child, an operation, etc.). The recommendation engine may use this information from the employee's application, as well as any available information corresponding to the employee's historical medical expenses over prior enrollment periods, as input to a machine learning model or artificial intelligence to predict the employee's medical expenses for the upcoming enrollment period and provide recommendations for establishing a HealthCredit line of credit, establishing and funding an HSA, selecting an optimal medical insurance plan for its needs, and the like. The machine learning model or artificial intelligence utilized by the recommendation engine may be trained using unsupervised learning techniques, as noted above.

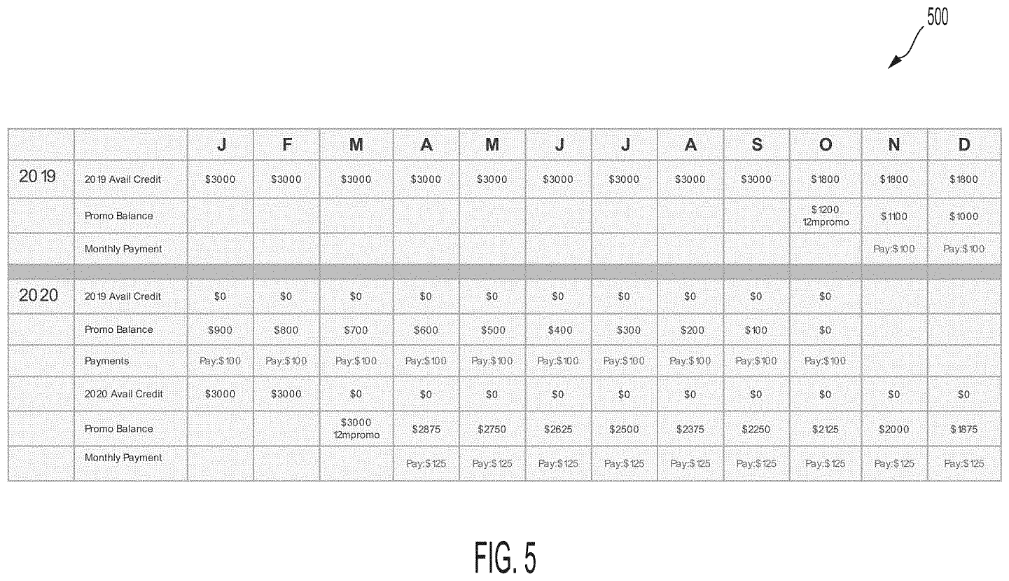

FIG. 5 depicts an exemplary table 500 of credit availability for an individual, according to some embodiments. The table shows available credit for the 2019 and 2020 years, a promotional balance, and a monthly payment. As shown, the promotional balance may be deducted from the available credit and annual spending caps reset at the start of the enrollment year. The table may be made available to an individual via a webservice implemented by the HealthCredit service. For instance, an individual may access a website or portal provided by the HealthCredit service and utilize its credentials to access the individual's HealthCredit account.

To generate the table, the HealthCredit service may access a cardholder database, as described above, to identify cardholder information of the individual. This cardholder information may specify the individual's present medical expenditures over the enrollment year or other enrollment period as defined by its employer. Further, the cardholder information may include data associated with the individual's current medical insurance plan, the individual's promotional benefits (e.g., reduced interest rates, bonus cash back rewards, etc.), any annual spending caps defined by the current medical insurance plan or employer, and the like. In some instances, the table may further specify any payments made by the employer on behalf of the individual from the individual's paycheck and/or HSA. Thus, the individual, via the website or portal provided by the HealthCredit service, may readily determine its medical expenses over an enrollment period, which the individual may use to influence its medical plan choices (e.g., selection of a plan, HSA contributions, etc.).

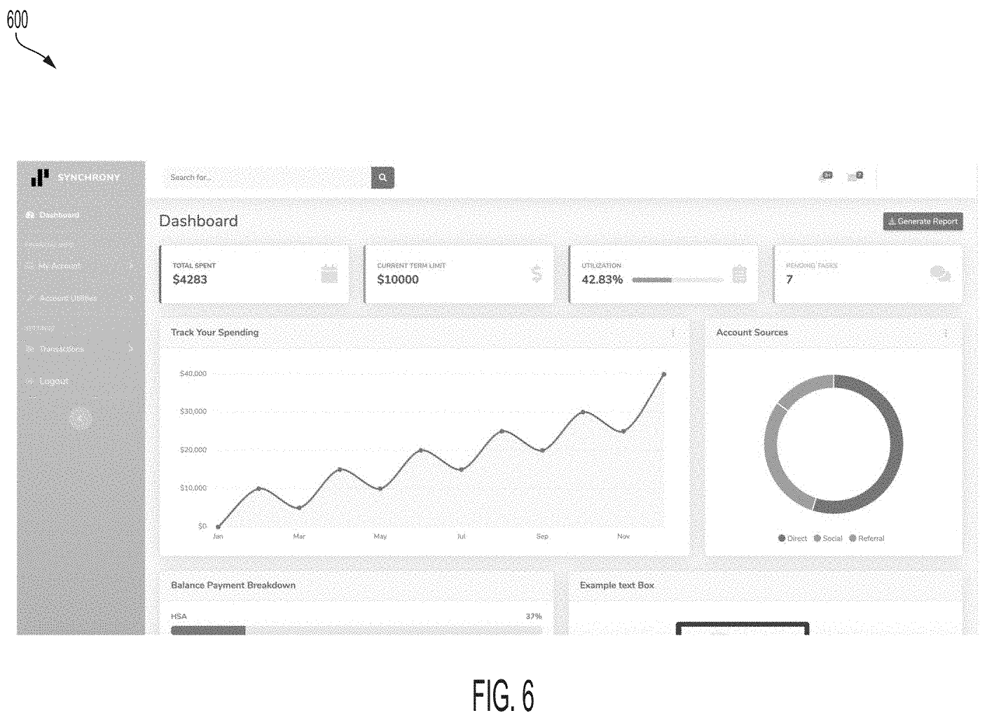

FIG. 6 depicts an exemplary interface 600 provided by the HealthCredit service to cardholders (e.g., employees of an employer that enables its employees to enroll in the HealthCredit service, former employees that may maintain a HealthCredit service account, etc.). The HealthCredit service, via the interface, may provide a cardholder with detailed information regarding their medical expenditures over an enrollment period. For example, as illustrated in FIG. 6, the HealthCredit service may indicate, using a graph or other informational device (e.g., charts, tables, etc.), the cardholder's usage of its medical plan benefits and of its line of credit with the HealthCredit service. Further, the HealthCredit service may indicate, via the interface, any spending caps imposed by its employer (e.g., “$10,000”) and the cardholder's current utilization of the line of credit with the HealthCredit service against this spending cap over the enrollment period (e.g., “$4283” or “42.83%”, as illustrated in FIG. 6). Thus, via the interface 600, a cardholder may readily determine its medical expenses over an enrollment period, its utilization of its medical plan benefits against any imposed spending caps, and the like. This information may be useful to the cardholder in determining whether to make adjustments to its medical plan coverage, its HSA contributions, and other needs.

To generate the information presented via the interface, the HealthCredit service may access the aforementioned employer database and cardholder database maintained by the HealthCredit service. As noted above, the employer database may include a list of employers, annual caps as defined by these employers, benefit years (e.g., enrollment periods, etc.), and promotional settings to be applied to each employee as defined by the employer. The information stored in the employer database may be provided by an employer when engaging the HealthCredit service to allow for enrollment of employees in the HealthCredit service as part of an enrollment period for medical benefits. For instance, as part of an employer enrollment process, the employer may be prompted to provide information regarding the medical insurance plans or other benefits that are provided to its employees, any spending caps that the employer may impose on its employees for medical expenses or any other contributions (e.g., retirement plan contributions, HSA contributions, etc.), its enrollment periods for different employee classes, and the like.

The cardholder database may specify information that is specific to each cardholder associated with the HealthCredit service. For instance, an entry within the cardholder database may correspond to a particular cardholder (e.g., employee or former employee of an employer, etc.). The entry may specify information specific to the cardholder, such as the cardholder's name, employer, salary, enrolled medical insurance plan, and the like. Further, the entry may specify information corresponding to the cardholder's line of credit with the HealthCredit service. This information may specify the amount of the line of credit, any outstanding balances, any payments made by the employer to the account, and the like. The HealthCredit service may utilize the information specified in the cardholder database in conjunction with the information from the employer database to generate the elements presented via the interface 600.

In an embodiment, the HealthCredit service may utilize a machine learning model or artificial intelligence trained using unsupervised learning techniques to provide an employee with a forecast of possible medical expenditures for the upcoming enrollment period, which may be presented via the interface 600. For instance, via the interface 600, the HealthCredit service may prompt an employee to indicate whether it is anticipating any events that may have an impact on the employee's medical expenditures over the upcoming enrollment period (e.g., birth of a child, an operation, etc.). The HealthCredit service may use this information, as well as any available information corresponding to the employee's historical medical expenses over prior enrollment periods, as input to a machine learning model or artificial intelligence to predict the employee's medical expenses for the upcoming enrollment period and provide recommendations for establishing a HealthCredit line of credit, establishing and funding an HSA, and the like. These recommendations may be provided to the employee via the interface 600 to allow the employee to readily compare different medical insurance plan options, to visualize the impact of any anticipated events on the employee's current and future medical expenditures, and the like.

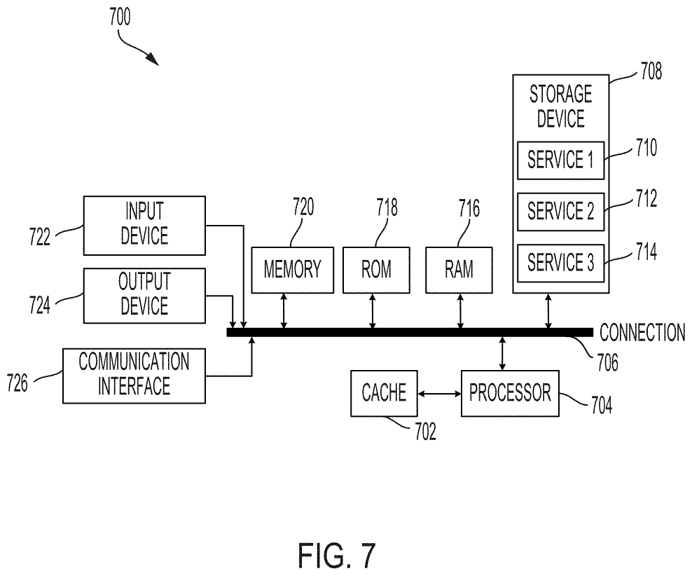

FIG. 7 illustrates a computing system architecture 700 including various components in electrical communication with each other using a connection 706, such as a bus, in accordance with some implementations. Example system architecture 700 includes a processing unit (CPU or processor) 704 and a system connection 706 that couples various system components including the system memory 720, such as ROM 718 and RAM 716, to the processor 704. The system architecture 700 can include a cache 702 of high-speed memory connected directly with, in close proximity to, or integrated as part of the processor 704. The system architecture 700 can copy data from the memory 720 and/or the storage device 708 to the cache 702 for quick access by the processor 704. In this way, the cache can provide a performance boost that avoids processor 704 delays while waiting for data. These and other modules can control or be configured to control the processor 704 to perform various actions.

Other system memory 720 may be available for use as well. The memory 720 can include multiple different types of memory with different performance characteristics. The processor 704 can include any general purpose processor and a hardware or software service, such as service 1 710, service 2 712, and service 3 714 stored in storage device 708, configured to control the processor 704 as well as a special-purpose processor where software instructions are incorporated into the actual processor design. The processor 704 may be a completely self-contained computing system, containing multiple cores or processors, a bus, memory controller, cache, etc. A multi-core processor may be symmetric or asymmetric.

To enable user interaction with the computing system architecture 700, an input device 722 can represent any number of input mechanisms, such as a microphone for speech, a touch-sensitive screen for gesture or graphical input, keyboard, mouse, motion input, speech and so forth. An output device 724 can also be one or more of a number of output mechanisms known to those of skill in the art. In some instances, multimodal systems can enable a user to provide multiple types of input to communicate with the computing system architecture 700. The communications interface 726 can generally govern and manage the user input and system output. There is no restriction on operating on any particular hardware arrangement and therefore the basic features here may easily be substituted for improved hardware or firmware arrangements as they are developed.

Storage device 708 is a non-volatile memory and can be a hard disk or other types of computer readable media which can store data that are accessible by a computer, such as magnetic cassettes, flash memory cards, solid state memory devices, digital versatile disks, cartridges, RAMs 716, ROM 718, and hybrids thereof.

The storage device 708 can include services 710, 712, 714 for controlling the processor 704. Other hardware or software modules are contemplated. The storage device 708 can be connected to the system connection 706. In one aspect, a hardware module that performs a particular function can include the software component stored in a computer-readable medium in connection with the necessary hardware components, such as the processor 704, connection 706, output device 724, and so forth, to carry out the function.

The disclosed methods can be performed using a computing system. An example computing system can include a processor (e.g., a central processing unit), memory, non-volatile memory, and an interface device. The memory may store data and/or and one or more code sets, software, scripts, etc. The components of the computer system can be coupled together via a bus or through some other known or convenient device. The processor may be configured to carry out all or part of methods described herein for example by executing code for example stored in memory. One or more of a user device or computer, a provider server or system, or a suspended database update system may include the components of the computing system or variations on such a system.

This disclosure contemplates the computer system taking any suitable physical form, including, but not limited to a Point-of-Sale system (“POS”). As example and not by way of limitation, the computer system may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, or a combination of two or more of these. Where appropriate, the computer system may include one or more computer systems; be unitary or distributed; span multiple locations; span multiple machines; and/or reside in a cloud, which may include one or more cloud components in one or more networks. Where appropriate, one or more computer systems may perform without substantial spatial or temporal limitation one or more steps of one or more methods described or illustrated herein. As an example and not by way of limitation, one or more computer systems may perform in real time or in batch mode one or more steps of one or more methods described or illustrated herein. One or more computer systems may perform at different times or at different locations one or more steps of one or more methods described or illustrated herein, where appropriate.

The processor may be, for example, be a conventional microprocessor such as an Intel Pentium microprocessor or Motorola power PC microprocessor. One of skill in the relevant art will recognize that the terms “machine-readable (storage) medium” or “computer-readable (storage) medium” include any type of device that is accessible by the processor.

The memory can be coupled to the processor by, for example, a bus. The memory can include, by way of example but not limitation, random access memory (RAM), such as dynamic RAM (DRAM) and static RAM (SRAM). The memory can be local, remote, or distributed.

The bus can also couple the processor to the non-volatile memory and drive unit. The non-volatile memory is often a magnetic floppy or hard disk, a magnetic-optical disk, an optical disk, a read-only memory (ROM), such as a CD-ROM, EPROM, or EEPROM, a magnetic or optical card, or another form of storage for large amounts of data. Some of this data is often written, by a direct memory access process, into memory during execution of software in the computer. The non-volatile storage can be local, remote, or distributed. The non-volatile memory is optional because systems can be created with all applicable data available in memory. A typical computer system will usually include at least a processor, memory, and a device (e.g., a bus) coupling the memory to the processor.

Software can be stored in the non-volatile memory and/or the drive unit. Indeed, for large programs, it may not even be possible to store the entire program in the memory. Nevertheless, it should be understood that for software to run, if necessary, it is moved to a computer readable location appropriate for processing, and for illustrative purposes, that location is referred to as the memory herein. Even when software is moved to the memory for execution, the processor can make use of hardware registers to store values associated with the software, and local cache that, ideally, serves to speed up execution. As used herein, a software program is assumed to be stored at any known or convenient location (from non-volatile storage to hardware registers), when the software program is referred to as “implemented in a computer-readable medium.” A processor is considered to be “configured to execute a program” when at least one value associated with the program is stored in a register readable by the processor.

The bus can also couple the processor to the network interface device. The interface can include one or more of a modem or network interface. It will be appreciated that a modem or network interface can be considered to be part of the computer system. The interface can include an analog modem, Integrated Services Digital network (ISDNO modem, cable modem, token ring interface, satellite transmission interface (e.g., “direct PC”), or other interfaces for coupling a computer system to other computer systems. The interface can include one or more input and/or output (I/O) devices. The I/O devices can include, by way of example but not limitation, a keyboard, a mouse or other pointing device, disk drives, printers, a scanner, and other input and/or output devices, including a display device. The display device can include, by way of example but not limitation, a cathode ray tube (CRT), liquid crystal display (LCD), or some other applicable known or convenient display device.

In operation, the computer system can be controlled by operating system software that includes a file management system, such as a disk operating system. One example of operating system software with associated file management system software is the family of operating systems known as Windows® from Microsoft Corporation of Redmond, Wash., and their associated file management systems. Another example of operating system software with its associated file management system software is the Linux™ operating system and its associated file management system. The file management system can be stored in the non-volatile memory and/or drive unit and can cause the processor to execute the various acts required by the operating system to input and output data and to store data in the memory, including storing files on the non-volatile memory and/or drive unit.

Some portions of the detailed description may be presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or “generating” or the like, refer to the action and processes of a computer system, or similar electronic computing device, that manipulates and transforms data represented as physical (electronic) quantities within registers and memories of the computer system into other data similarly represented as physical quantities within the computer system memories or registers or other such information storage, transmission or display devices.

The algorithms and displays presented herein are not inherently related to any particular computer or other apparatus. Various general purpose systems may be used with programs in accordance with the teachings herein, or it may prove convenient to construct more specialized apparatus to perform the methods of some examples. The required structure for a variety of these systems will appear from the description below. In addition, the techniques are not described with reference to any particular programming language, and various examples may thus be implemented using a variety of programming languages.

In various implementations, the system operates as a standalone device or may be connected (e.g., networked) to other systems. In a networked deployment, the system may operate in the capacity of a server or a client system in a client-server network environment, or as a peer system in a peer-to-peer (or distributed) network environment.

The system may be a server computer, a client computer, a personal computer (PC), a tablet PC, a laptop computer, a set-top box (STB), a personal digital assistant (PDA), a cellular telephone, an iPhone, a Blackberry, a processor, a telephone, a web appliance, a network router, switch or bridge, or any system capable of executing a set of instructions (sequential or otherwise) that specify actions to be taken by that system.

While the machine-readable medium or machine-readable storage medium is shown, by way of example, to be a single medium, the term “machine-readable medium” and “machine-readable storage medium” should be taken to include a single medium or multiple media (e.g., a centralized or distributed database, and/or associated caches and servers) that store the one or more sets of instructions. The term “machine-readable medium” and “machine-readable storage medium” shall also be taken to include any medium that is capable of storing, encoding, or carrying a set of instructions for execution by the system and that cause the system to perform any one or more of the methodologies or modules of disclosed herein.

In general, the routines executed to implement the implementations of the disclosure, may be implemented as part of an operating system or a specific application, component, program, object, module or sequence of instructions referred to as “computer programs.” The computer programs typically comprise one or more instructions set at various times in various memory and storage devices in a computer, and that, when read and executed by one or more processing units or processors in a computer, cause the computer to perform operations to execute elements involving the various aspects of the disclosure.

Moreover, while examples have been described in the context of fully functioning computers and computer systems, those skilled in the art will appreciate that the various examples are capable of being distributed as a program object in a variety of forms, and that the disclosure applies equally regardless of the particular type of machine or computer-readable media used to actually effect the distribution.

Further examples of machine-readable storage media, machine-readable media, or computer-readable (storage) media include but are not limited to recordable type media such as volatile and non-volatile memory devices, floppy and other removable disks, hard disk drives, optical disks (e.g., Compact Disk Read-Only Memory (CD ROMS), Digital Versatile Disks, (DVDs), etc.), among others, and transmission type media such as digital and analog communication links.

In some circumstances, operation of a memory device, such as a change in state from a binary one to a binary zero or vice-versa, for example, may comprise a transformation, such as a physical transformation. With particular types of memory devices, such a physical transformation may comprise a physical transformation of an article to a different state or thing. For example, but without limitation, for some types of memory devices, a change in state may involve an accumulation and storage of charge or a release of stored charge. Likewise, in other memory devices, a change of state may comprise a physical change or transformation in magnetic orientation or a physical change or transformation in molecular structure, such as from crystalline to amorphous or vice versa. The foregoing is not intended to be an exhaustive list of all examples in which a change in state for a binary one to a binary zero or vice-versa in a memory device may comprise a transformation, such as a physical transformation. Rather, the foregoing is intended as illustrative examples.

A storage medium typically may be non-transitory or comprise a non-transitory device. In this context, a non-transitory storage medium may include a device that is tangible, meaning that the device has a concrete physical form, although the device may change its physical state. Thus, for example, non-transitory refers to a device remaining tangible despite this change in state.

The above description and drawings are illustrative and are not to be construed as limiting the subject matter to the precise forms disclosed. Persons skilled in the relevant art can appreciate that many modifications and variations are possible in light of the above disclosure. Numerous specific details are described to provide a thorough understanding of the disclosure. However, in certain instances, well-known or conventional details are not described in order to avoid obscuring the description.

As used herein, the terms “connected,” “coupled,” or any variant thereof when applying to modules of a system, means any connection or coupling, either direct or indirect, between two or more elements; the coupling of connection between the elements can be physical, logical, or any combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, or any combination of the items in the list.

Those of skill in the art will appreciate that the disclosed subject matter may be embodied in other forms and manners not shown below. It is understood that the use of relational terms, if any, such as first, second, top and bottom, and the like are used solely for distinguishing one entity or action from another, without necessarily requiring or implying any such actual relationship or order between such entities or actions.

While processes or blocks are presented in a given order, alternative implementations may perform routines having steps, or employ systems having blocks, in a different order, and some processes or blocks may be deleted, moved, added, subdivided, substituted, combined, and/or modified to provide alternative or sub combinations. Each of these processes or blocks may be implemented in a variety of different ways. Also, while processes or blocks are at times shown as being performed in series, these processes or blocks may instead be performed in parallel, or may be performed at different times. Further any specific numbers noted herein are only examples: alternative implementations may employ differing values or ranges.

The teachings of the disclosure provided herein can be applied to other systems, not necessarily the system described above. The elements and acts of the various examples described above can be combined to provide further examples.

Any patents and applications and other references noted above, including any that may be listed in accompanying filing papers, are incorporated herein by reference. Aspects of the disclosure can be modified, if necessary, to employ the systems, functions, and concepts of the various references described above to provide yet further examples of the disclosure.

These and other changes can be made to the disclosure in light of the above Detailed Description. While the above description describes certain examples, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific implementations disclosed in the specification, unless the above Detailed Description section explicitly defines such terms. Accordingly, the actual scope of the disclosure encompasses not only the disclosed implementations, but also all equivalent ways of practicing or implementing the disclosure under the claims.

While certain aspects of the disclosure are presented below in certain claim forms, the inventors contemplate the various aspects of the disclosure in any number of claim forms. Any claims intended to be treated under 35 U.S.C. § 112(f) will begin with the words “means for”. Accordingly, the applicant reserves the right to add additional claims after filing the application to pursue such additional claim forms for other aspects of the disclosure.

The terms used in this specification generally have their ordinary meanings in the art, within the context of the disclosure, and in the specific context where each term is used. Certain terms that are used to describe the disclosure are discussed above, or elsewhere in the specification, to provide additional guidance to the practitioner regarding the description of the disclosure. For convenience, certain terms may be highlighted, for example using capitalization, italics, and/or quotation marks. The use of highlighting has no influence on the scope and meaning of a term; the scope and meaning of a term is the same, in the same context, whether or not it is highlighted. It will be appreciated that same element can be described in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various examples given in this specification.

Without intent to further limit the scope of the disclosure, examples of instruments, apparatus, methods and their related results according to the examples of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions will control.

Some portions of this description describe examples in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In some examples, a software module is implemented with a computer program object comprising a computer-readable medium containing computer program code, which can be executed by a computer processor for performing any or all of the steps, operations, or processes described.

Examples may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Examples may also relate to an object that is produced by a computing process described herein. Such an object may comprise information resulting from a computing process, where the information is stored on a non-transitory, tangible computer readable storage medium and may include any implementation of a computer program object or other data combination described herein.

The language used in the specification has been principally selected for readability and instructional purposes, and it may not have been selected to delineate or circumscribe the subject matter. It is therefore intended that the scope of this disclosure be limited not by this detailed description, but rather by any claims that issue on an application based hereon. Accordingly, the disclosure of the examples is intended to be illustrative, but not limiting, of the scope of the subject matter, which is set forth in the following claims.

Specific details were given in the preceding description to provide a thorough understanding of various implementations of systems and components for a contextual connection system. It will be understood by one of ordinary skill in the art, however, that the implementations described above may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

It is also noted that individual implementations may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination can correspond to a return of the function to the calling function or the main function.

Client devices, network devices, and other devices can be computing systems that include one or more integrated circuits, input devices, output devices, data storage devices, and/or network interfaces, among other things. The integrated circuits can include, for example, one or more processors, volatile memory, and/or non-volatile memory, among other things. The input devices can include, for example, a keyboard, a mouse, a key pad, a touch interface, a microphone, a camera, and/or other types of input devices. The output devices can include, for example, a display screen, a speaker, a haptic feedback system, a printer, and/or other types of output devices. A data storage device, such as a hard drive or flash memory, can enable the computing device to temporarily or permanently store data. A network interface, such as a wireless or wired interface, can enable the computing device to communicate with a network. Examples of computing devices include desktop computers, laptop computers, server computers, hand-held computers, tablets, smart phones, personal digital assistants, digital home assistants, as well as machines and apparatuses in which a computing device has been incorporated.

The term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as compact disk (CD) or digital versatile disk (DVD), flash memory, memory or memory devices. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like.

The various examples discussed above may further be implemented by hardware, software, firmware, middleware, microcode, hardware description languages, or any combination thereof. When implemented in software, firmware, middleware or microcode, the program code or code segments to perform the necessary tasks (e.g., a computer-program product) may be stored in a computer-readable or machine-readable storage medium (e.g., a medium for storing program code or code segments). A processor(s), implemented in an integrated circuit, may perform the necessary tasks.

Where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

The various illustrative logical blocks, modules, circuits, and algorithm steps described in connection with the implementations disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the methods described above. The computer-readable data storage medium may form part of a computer program product, which may include packaging materials. The computer-readable medium may comprise memory or data storage media, such as random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general purpose processor may be a microprocessor; but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein. In addition, in some aspects, the functionality described herein may be provided within dedicated software modules or hardware modules configured for implementing a suspended database update system.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claim.

What is claimed is:

1. A computer-implemented method comprising:
receiving a request for credit from a patient, wherein the credit is used to pay for a healthcare service, and wherein repayment of the credit is performed by an employer associated with the patient;
calculating a spending limit for the patient, wherein calculating the spending limit includes retrieving a paycheck for the patient from the employer associated with the patient and accessing underwriting data to identify a maximum percentage of the paycheck that can be allocated to the healthcare service;
calculating a repayment schedule for the credit, wherein the repayment schedule includes a minimum monthly payment amount toward the spending limit and a duration of time to pay back the credit; and
receiving repayment of the credit from the employer according to the repayment schedule.

2. The computer-implemented method of claim 1, further comprising:
detecting access to a medical plan associated with the healthcare service, wherein the access is detected from a point-of-sale associated with the healthcare service;
determining a balance for the healthcare service, wherein the balance is determined based on one or more deductibles associated with the medical plan;
identifying an amount available from a health saving account (HSA) associated with the patient for payment of healthcare services; and
prompting the patient to provide payment for the remaining balance using any combination of the amount available from the HSA, the credit, and other available payment methods.

3. The computer-implemented method of claim 1, further comprising:
detecting use of the credit to pay for the healthcare service; and
transmitting a bill indicating a payment amount for the use of the credit to the employer to cause the employer to repay the payment amount.

4. The computer-implemented method of claim 1, wherein the credit and medical plan information associated with the patient are implemented on a payment instrument provided to the patient.

5. The computer-implemented method of claim 1, wherein the spending limit for the patient is set to a maximum deductible amount of a medical plan associated with the patient, wherein the maximum deductible amount does not exceed an amount corresponding to the maximum percentage.

6. The computer-implemented method of claim 1, wherein calculating the spending limit further includes performing a credit worthiness check associated with the patient to determine a credit amount allocatable to the patient.

7. The computer-implemented method of claim 1, wherein the request for the credit from the patient is received with a request for enrollment in a medical plan from the patient.

8. A system, comprising:
one or more processors; and
memory storing thereon instructions that, as a result of being executed by the one or more processors, cause the system to:
receive a request for credit from a patient, wherein the credit is used to pay for a healthcare service, and wherein repayment of the credit is performed by an employer associated with the patient;
calculate a spending limit for the patient, wherein calculating the spending limit includes retrieving a paycheck for the patient from the employer associated with the patient and accessing underwriting data to identify a maximum percentage of the paycheck that can be allocated to the healthcare service;
calculate a repayment schedule for the credit, wherein the repayment schedule includes a minimum monthly payment amount toward the spending limit and a duration of time to pay back the credit; and
receive repayment of the credit from the employer according to the repayment schedule.

9. The system of claim 8, wherein the instructions further cause the system to:
detect access to a medical plan associated with the healthcare service, wherein the access is detected from a point-of-sale associated with the healthcare service;
determine a balance for the healthcare service, wherein the balance is determined based on one or more deductibles associated with the medical plan;
identify an amount available from a health saving account (HSA) associated with the patient for payment of healthcare services; and
prompt the patient to provide payment for the remaining balance using any combination of the amount available from the HSA, the credit, and other available payment methods.

10. The system of claim 8, wherein the instructions further cause the system to:
detect use of the credit to pay for the healthcare service; and
transmit a bill indicating a payment amount for the use of the credit to the employer to cause the employer to repay the payment amount.

11. The system of claim 8, wherein the credit and medical plan information associated with the patient are implemented on a payment instrument provided to the patient.

12. The system of claim 8, wherein the spending limit for the patient is set to a maximum deductible amount of a medical plan associated with the patient, wherein the maximum deductible amount does not exceed an amount corresponding to the maximum percentage.

13. The system of claim 8, wherein the instructions that cause the system to calculate the spending limit further cause the system to perform a credit worthiness check associated with the patient to determine a credit amount allocatable to the patient.

14. The system of claim 8, wherein the request for the credit from the patient is received with a request for enrollment in a medical plan from the patient.

15. A non-transitory, computer-readable storage medium storing thereon executable instructions that, as a result of being executed by one or more processors of a computer system, cause the computer system to:
receive a request for credit from a patient, wherein the credit is used to pay for a healthcare service, and wherein repayment of the credit is performed by an employer associated with the patient;
calculate a spending limit for the patient, wherein calculating the spending limit includes retrieving a paycheck for the patient from the employer associated with the patient and accessing underwriting data to identify a maximum percentage of the paycheck that can be allocated to the healthcare service;
calculate a repayment schedule for the credit, wherein the repayment schedule includes a minimum monthly payment amount toward the spending limit and a duration of time to pay back the credit; and
receive repayment of the credit from the employer according to the repayment schedule.

16. The non-transitory, computer-readable storage medium of claim 15, wherein the executable instructions further cause the computer system to:
detect access to a medical plan associated with the healthcare service, wherein the access is detected from a point-of-sale associated with the healthcare service;
determine a balance for the healthcare service, wherein the balance is determined based on one or more deductibles associated with the medical plan;
identify an amount available from a health saving account (HSA) associated with the patient for payment of healthcare services; and
prompt the patient to provide payment for the remaining balance using any combination of the amount available from the HSA, the credit, and other available payment methods.

17. The non-transitory, computer-readable storage medium of claim 15, wherein the executable instructions further cause the computer system to:
detect use of the credit to pay for the healthcare service; and
transmit a bill indicating a payment amount for the use of the credit to the employer to cause the employer to repay the payment amount.

18. The non-transitory, computer-readable storage medium of claim 15, wherein the spending limit for the patient is set to a maximum deductible amount of a medical plan associated with the patient, wherein the maximum deductible amount does not exceed an amount corresponding to the maximum percentage.

19. The non-transitory, computer-readable storage medium of claim 15, wherein the executable instructions that cause the computer system to calculate the spending limit further cause the computer system to perform a credit worthiness check associated with the patient to determine a credit amount allocatable to the patient.

20. The non-transitory, computer-readable storage medium of claim 15, wherein the request for the credit from the patient is received with a request for enrollment in a medical plan from the patient.
